(19) United States
(12) Patent Application Publication
(10) Pub. No.: US 2026/0146797 A1
(43) Pub. Date: May 28, 2026

(54) ZERO-CARBON GLASS FURNACE PROCESS

(71) Applicant: SHANGHAI YUANHAN ENERGY & CHEMICAL TECHNOLOGY CO., LTD., Shanghai (CN)

(72) Inventors: Yunfeng ZHANG, Shanghai (CN); Xiangquan ZHANG, Shanghai (CN); Wenjun WU, Shanghai (CN)

(73) Assignee: SHANGHAI YUANHAN ENERGY & CHEMICAL TECHNOLOGY CO., LTD., Shanghai (CN)

(21) Appl. No.: 19/312,358

(22) Filed: Aug. 28, 2025

Related U.S. Application Data
(63) Continuation-in-part of application No. PCT/CN2025/080794, filed on Mar. 5, 2025.

(30) Foreign Application Priority Data
Nov. 22, 2024 (CN) 202411679279.0

(51) Int. Cl. F27D 17/15; B01D 53/52; C03B 5/235; F27D 17/10; F27D 17/20; F27D 99/00

(52) U.S. Cl. CPC F27D 17/15; B01D 53/52; C03B 5/235; F27D 17/102; F27D 17/20; B01D 2256/22; B01D 2257/304; B01D 2258/0241; C03B 2211/40; F27D 2099/005

(57) ABSTRACT

Provided is a zero-carbon glass furnace process, a system required for the process includes a photovoltaic power generation unit, an air separation unit, a water electrolysis hydrogen production unit, a mixer, a methanation unit, a first waste heat boiler, a reforming unit, a glass furnace, a second waste heat boiler, and a dedusting and desulfurization unit. The process adopts green electricity to produce oxygen, nitrogen and hydrogen; flue gas of the glass furnace is circularly enriched into a high concentration of carbon dioxide, which is methanized with hydrogen to produce methane, and the methane is reformed with carbon dioxide and water vapor to produce carbon monoxide and hydrogen; the carbon monoxide and hydrogen are used as fuel of the glass furnace, the oxygen and carbon dioxide are mixed into carbon-based enriched oxygen as a combustion aid of the glass furnace, and the excess carbon dioxide is sold externally.

### CROSS-REFERENCE TO RELATED APPLICATIONS

This application is a continuation-in-part application of International Application No. PCT/CN2025/080794 filed on Mar. 5, 2025, which claims priority to Chinese Patent Application No. 202411679279.0, filed on Nov. 22, 2024, the entire contents of each of which are hereby incorporated by reference.

### TECHNICAL FIELD

The present disclosure relates to the technical field of glass furnace, and in particular, to a zero-carbon glass furnace process.

### BACKGROUND

Greenhouse gas emissions are the most important factor causing global warming. The greenhouse effect caused by carbon dioxide accounts for more than 70 v % of that from all greenhouse gases, so carbon dioxide emission reduction is an urgent problem to be solved, which is important for controlling the greenhouse effect and slowing down global warming.

Currently, flue gas from glass furnace is basically discharged directly into atmosphere. Although carbon emission share of glass industry is not large, it is a high-energy-consumption and high-emission industry, and CO₂ emissions are not optimistic.

Therefore, a zero-carbon glass furnace process is provided to address the deficiencies of the prior art.

### SUMMARY

According to some embodiments of the present disclosure, a zero-carbon glass furnace process is provided. A system required for the process includes a photovoltaic power generation unit, an air separation unit, a water electrolysis hydrogen production unit, a mixer, a methanation unit, a first waste heat boiler, a reforming unit, a glass furnace, a second waste heat boiler, and a dedusting and desulfurization unit. The photovoltaic power generation unit is configured to produce green electricity to supply electricity to the air separation unit and the water electrolysis hydrogen production unit. The air separation unit is configured to produce oxygen and nitrogen. The water electrolysis hydrogen production unit is configured to produce hydrogen and oxygen. The mixer is configured to mix the oxygen from the air separation unit, the oxygen from the water electrolysis hydrogen production unit, and circulating flue gas from the glass furnace into carbon-based enriched oxygen. The methanation unit is configured to methanize the hydrogen from the water electrolysis hydrogen production unit and circulating flue gas from the dedusting and desulfurization unit. The first waste heat boiler is configured to recover waste heat of methane containing saturated water vapor from the methanation unit and by-produce steam. The reforming unit is configured to subject the methane containing saturated water vapor from the first waste heat boiler and a portion of the circulating flue gas from the glass furnace to a methane reforming reaction. The glass furnace is configured to produce molten glass using carbon monoxide and hydrogen from the reforming unit as fuel and the carbon-based enriched oxygen from the mixer as a combustion aid. The second waste heat boiler is configured to recover waste heat of the circulating flue gas from the glass furnace and by-produce steam. The dedusting and desulfurization unit is configured to dedust and desulfurize circulating flue gas from the second waste heat boiler. The photovoltaic power generation unit is connected to the air separation unit and the water electrolysis hydrogen production unit, respectively; a nitrogen outlet of the air separation unit is connected to a tin bath of the glass furnace and to a nitrogen product storage tank, respectively, and a pressure reducing valve is provided on a connecting pipe between the nitrogen outlet of the air separation unit and the tin bath of the glass furnace; an oxygen outlet of the air separation unit and an oxygen outlet of the water electrolysis hydrogen production unit are both connected to the mixer, a pressure reducing valve is provided on a connecting pipe between the oxygen outlet of the water electrolysis hydrogen production unit and the mixer, a connecting pipe between the oxygen outlet of the air separation unit, the oxygen outlet of the water electrolysis hydrogen production unit, and the mixer is provided with a flow detector, a temperature detector, a pressure detector, an oxygen purity detector, and a flow regulating valve; a hydrogen outlet of the water electrolysis hydrogen production unit is connected to the methanation unit; a carbon-based enriched oxygen outlet of the mixer is switchably connected to a heat storage grid A/B of the glass furnace and then connected to the glass furnace; a methane outlet of the methanation unit is connected to the first waste heat boiler; a methane outlet of the first waste heat boiler is switchably connected to a heat storage grid A/B of the reforming unit and then connected to the reforming unit, a pressure reducing valve is provided on a connecting pipe between the methane outlet of the first waste heat boiler and the heat storage grid A/B of the reforming unit, and a steam outlet of the first waste heat boiler is connected to a steam utilizer; a carbon monoxide and hydrogen outlet of the reforming unit is connected to the glass furnace; one of circulating flue gas outlets of the glass furnace is switchably connected to the heat storage grid B/A of the glass furnace and then connected to the second waste heat boiler and the mixer, one of the circulating flue gas outlets of the glass furnace is switchably connected to the heat storage grid A/B of the reforming unit and then connected to the reforming unit, and one of the circulating flue gas outlets of the glass furnace is switchably connected to a heat storage grid B/A of the reforming unit and then connected to the dedusting and desulfurization unit, respectively, a pressure booster is provided on a connecting pipe between the heat storage grid B/A of the glass furnace, the second waste heat boiler, and the mixer, a connecting pipe between the heat storage grid B/A of the glass furnace and the mixer is provided with a flow detector, a temperature detector, a pressure detector, a carbon dioxide purity detector, and a flow regulating valve, a pressure booster is provided on the connecting pipe between the glass furnace, the heat storage grid A/B of the reforming unit, and the heat storage grid B/A of the reforming unit, and a pressure reducing valve is provided on a connecting pipe between the heat storage grid B/A of the reforming unit and the dedusting and desulfurization unit; a circulating flue gas outlet of the second waste heat boiler is connected to the dedusting and desulfurization unit; circulating flue gas outlets of the dedusting and desulfurization unit are connected to the methanation unit and a carbon dioxide product storage tank, respectively, and a pressure booster is provided on a connecting pipe between one of the circulating flue gas outlets of the dedusting and desulfurization unit and the methanation unit.

The process comprises operations of: (1) the photovoltaic power generation unit producing the green electricity to supply the electricity for the air separation unit and the water electrolysis hydrogen production unit; the air separation unit producing oxygen and nitrogen with the green electricity supplied by the photovoltaic power generation unit as the electricity, wherein the oxygen is fed into the mixer, a portion of the nitrogen is depressurized and then fed into the tin bath of the glass furnace as protective gas, and the rest is sold outside; the water electrolysis hydrogen production unit producing hydrogen and oxygen with the green electricity supplied by the photovoltaic power generation unit as the electricity, wherein the hydrogen is fed into the methanation unit, and the oxygen is fed into the mixer after depressurization; (2) the mixer mixing the oxygen from the air separation unit, the oxygen from the water electrolysis hydrogen production unit, and the circulating flue gas from the glass furnace into the carbon-based enriched oxygen, and using the carbon-based enriched oxygen as the combustion aid for the glass furnace; (3) the methanation unit methanizing the hydrogen from the water electrolysis hydrogen production unit and the circulating flue gas from the dedusting and desulfurization unit to produce the methane containing saturated water vapor, and feeding the methane containing saturated water vapor into the first waste heat boiler; (4) the first waste heat boiler recovering the waste heat of the methane containing saturated water vapor from the methanation unit and by-producing the steam; selling the steam externally, and feeding depressurized methane containing saturated water vapor after waste heat recovery to the reforming unit; (5) the reforming unit subjecting the methane containing saturated water vapor from the first waste heat boiler and the portion of the circulating flue gas from the glass furnace to the methane reforming reaction; the circulating flue gas from the glass furnace entering into the reforming unit in two ways, one way entering into a heat storage grid of one side of the reforming unit, providing heat for the heat storage grid of the one side of the reforming unit to provide heat for raw gas of the reforming unit in a next round, and after heat exchange, the circulating flue gas being depressurized and fed into the dedusting and desulfurization unit; the other way entering into a heat storage grid on the other side of the reforming unit along with the methane containing saturated water vapor from the first waste heat boiler, the heat storage grid on the other side of the reforming unit being already heated by the circulating flue gas of a previous round, two sides of the heat storage grids working in rotation, and the raw gas fed into the reforming unit being heated by heat of the heat storage grid on the other side of the reforming unit, in a reaction bed of the reforming unit, under a certain pressure and a certain catalyst, methane, carbon dioxide, and water vapor undergoing the methane reforming reaction to produce the carbon monoxide and the hydrogen, and feeding the carbon monoxide and the hydrogen into the glass furnace as the fuel; (6) the glass furnace producing the molten glass using the carbon monoxide and the hydrogen from the reforming unit as the fuel and the carbon-based enriched oxygen from the mixer as the combustion aid; the carbon-based enriched oxygen from the mixer first passing through a heat storage grid on one side of the glass furnace, the heat storage grid on the one side of the glass furnace being already heated by the previous round of the circulating flue gas, two sides of the heat storage grids working in rotation, and the carbon-based enriched oxygen from the mixer being heated to a certain temperature and then fed into the glass furnace; after the flue gas of the glass furnace is circularly enriched for a certain period of time, and a carbon dioxide concentration reaching an equilibrium; the circulating flue gas being divided into two ways, one way passing through a heat storage grid on the other side of the glass furnace, providing heat for the heat storage grid on the other side of the glass furnace to provide heat for the carbon-based enriched oxygen from the mixer in the next round, and after heat exchange, the circulating flue gas of this way being pressurized and fed into the mixer and the second waste heat boiler; the other way not passing through the heat storage grid of the glass furnace, and being pressurized and fed into the reforming unit; (7) the second waste heat boiler recovering the waste heat of the circulating flue gas from the glass furnace and by-producing the steam; selling the steam externally, and feeding the circulating flue gas after waste heat recovery to the dedusting and desulfurization unit; and (8) the dedusting and desulfurization unit dedusting and desulfurizing the circulating flue gas from the second waste heat boiler; pressurizing a portion of circulating flue gas after dedusting and desulfurizing and feeding pressurized circulating flue gas into the methanation unit, and selling the rest externally after cooled down as carbon dioxide products; wherein in an initial stage, the glass furnace uses natural gas as the fuel and air as the combustion aid, and after flue gas is produced, the carbon-based enriched oxygen mixed by the flue gas, the oxygen produced by the air separation unit, and the oxygen produced by the water electrolysis hydrogen production unit gradually replaces the air and is used as the combustion aid, after a certain period of cyclic enrichment, the carbon dioxide concentration of the circulating flue gas reaches the equilibrium, and the methanation unit, the reforming unit, the first waste heat boiler, the second waste heat boiler, and the dedusting and desulfurization unit are put into operation, and the system enters into a normal operation state.

In some embodiments, in operation (1), the oxygen produced by the air separation unit has a purity of 99.6 v % or higher, a normal temperature, and a pressure of 0.2-0.3 MPa, and the oxygen is fed into the mixer; the nitrogen produced by the air separation unit has a purity of 99.9 v % or higher, a normal temperature, and a pressure of 0.2-0.3 MPa, the portion of the nitrogen is depressurized to 0.05 MPa and fed into the tin bath of the glass furnace as the protective gas, and the rest is sold externally; and the hydrogen produced by the water electrolysis hydrogen production unit has a purity of 99.8 v % or higher, a temperature of 90-100° C., and a pressure of 2.5-3.0 MPa, and the hydrogen is fed into the methanation unit; the oxygen produced by the water electrolysis hydrogen production unit has a purity of 99.0 v % or higher, a temperature of 90-100° C., and a pressure of 2.5-3.0 MPa, and the oxygen is depressurized to 0.2-0.3 MPa and fed into the mixer.

In some embodiments, in operation (2), the oxygen from the air separation unit has a purity of 99.6 v % or higher, a normal temperature, and a pressure of 0.2-0.3 MPa; the oxygen from the water electrolysis hydrogen production unit has a purity of 99.0 v % or higher, a temperature of 90-100° C., and a pressure of 0.2-0.3 MPa; the circulating flue gas from the glass furnace has a carbon dioxide concentration of 95.0 v % or higher, a temperature of 550-650° C., and a pressure of 0.15-0.25 MPa; the oxygen from the air separation unit, the oxygen from the water electrolysis hydrogen production unit, and the circulating flue gas from the glass furnace are each regulated in flow rate via flow regulating valves, the carbon-based enriched oxygen has an oxygen content of 21-30 v %, a temperature of 460-480° C., and a pressure of 0.05-0.10 MPa, and the oxygen content is adjusted according to different requirements of the glass furnace.

In some embodiments, in operation (3), the hydrogen from the water electrolysis hydrogen production unit has a purity of 99.8 v % or higher, a temperature of 90-100° C., and a pressure of 2.5-3.0 MPa, the circulating flue gas from the dedusting and desulfurization unit has a carbon dioxide concentration of 95.0 v % or higher, a temperature of 250-300° C., and a pressure of 2.5-3.0 MPa, the hydrogen and the circulating flue gas are fed into the methanation unit at a volume ratio of H₂ and CO₂ of 4:1, under an action of a nickel-based methanation catalyst, the hydrogen and the carbon dioxide are catalyzed to generate methane, while releasing a large amount of heat, the methane has a purity of 94.0 v % or higher on a dry basis, a temperature of 390-410° C., a pressure of 2.5-3.0 MPa, and the methane containing saturated water vapor is fed into the first waste heat boiler.

In some embodiments, in operation (4), the methane containing saturated water vapor from the methanation unit has a temperature of 390-410° C., the temperature is lowered to 240-260° C. after the waste heat recovery by the first waste heat boiler and steam with a pressure of 1.5 MPa is by-produced for external sale; and the methane containing saturated water vapor after the waste heat recovery is depressurized to 0.5-1.0 MPa and then fed into the reforming unit.

In some embodiments, in operation (5), the methane containing saturated water vapor from the first waste heat boiler has a methane purity of 94.0 v % or higher on a dry basis, a temperature of 240-260° C., and a pressure of 0.5-1.0 MPa, and the circulating flue gas from the glass furnace has a carbon dioxide concentration of 95.0 v % or higher, a temperature of 1300-1400° C., and a pressure of 0.5-1.0 MPa; the circulating flue gas from the glass furnace is divided into two ways to enter the reforming unit, one way enters the heat storage grid on the one side of the reforming unit, providing heat for the heat storage grid on the one side of the reforming unit to provide heat for the raw gas of the reforming unit in the next round, after the heat exchange, the temperature of the circulating flue gas is reduced to 250-300° C., and the pressure of the circulating flue gas is reduced to 0.1-0.2 MPa and then fed into the dedusting and desulfurization unit; the other way and the methane containing saturated water vapor from the first waste heat boiler are fed into the heat storage grid on the other side of the reforming unit at a volume ratio of CH₄ and CO₂ of 2:1, the heat storage grid on the other side of the reforming unit is heated by the circulating flue gas of the previous round, the two heat storage grids work in rotation, and the raw gas fed into the reforming unit is heated to 900-1000° C. by the heat storage grid on the other side of the reforming unit, under a pressure of 0.5-1.0 MPa and a nickel-based catalyst, the methane, the carbon dioxide, and the water vapor undergo the methane reforming reaction to produce the carbon monoxide and the hydrogen, with a volume ratio of CO and H₂ of 3:5, an outlet temperature of 700-800° C., and a pressure of 0.5-1.0 MPa, and the carbon monoxide and the hydrogen are fed into the glass furnace as the fuel.

In some embodiments, in operation (6), the carbon monoxide and the hydrogen from the reforming unit have a volume ratio of CO and H₂ of 3:5, a temperature of 700-800° C., and a pressure of 0.5-1.0 MPa, and the carbon-based enriched oxygen from the mixer has an oxygen content of 21-30 v %, a temperature of 460-480° C., and a pressure of 0.05-0.10 MPa; the carbon-based enriched oxygen from the mixer first passes through the heat storage grid of the one side of the glass furnace, the heat storage grid is heated by the previous round of circulating flue gas, the two sides of the heat storage grids work in rotation, and the carbon-based enriched oxygen from the mixer is heated to 700-800° C. and then fed into the glass furnace; the fuel and the combustion aid have a volume ratio of 1:2; in the glass furnace, under a combustion of the carbon-based enriched oxygen, the carbon monoxide and the hydrogen undergo a combustion reaction, providing heat for the production of the molten glass in the glass furnace; and after the flue gas of the glass furnace is circularly enriched for 3-5 hours, and the carbon dioxide concentration reaches the equilibrium, with the carbon dioxide concentration of 95.0 v % or higher, a temperature of 1300-1400° C., and a pressure of 0.01-0.05 MPa; the circulating flue gas is divided into two ways, one way passes through the heat storage grid on the other side of the glass furnace, providing heat for the heat storage grid on the other side of the glass furnace to provide heat for the carbon-based enriched oxygen from the mixer in the next round, after the heat exchange, the circulating flue gas of this way is cooled down to 550-650° C. and pressurized to 0.15-0.25 MPa, and the circulating flue gas is fed into the mixer and the second waste heat boiler; the other way does not pass through the heat storage grid of the glass furnace, with a temperature of 1300-1400° C., is pressurized to 0.5-1.0 MPa, and fed into the reforming unit.

In some embodiments, in operation (7), the circulating flue gas from the glass furnace has a carbon dioxide concentration of 95.0 v % or higher, a temperature of 550-650° C., and a pressure of 0.15-0.25 MPa, after the waste heat recovery by the second waste heat boiler, the temperature is reduced to 250-300° C., and the pressure is 0.1-0.2 MPa, and the steam with a pressure of 1.5-4.0 MPa is by-produced for external sale; and the circulating flue gas after the waste heat recovery is fed into the dedusting and desulfurization unit.

In some embodiments, the dedusting and desulfurization unit includes a high-temperature dust collector and a dry desulfurization device; the high-temperature dust collector adopts cyclone separating and dust removing, and the dry desulfurization device adopts zinc oxide desulfurization. In operation (8), the circulating flue gas first passes through the high-temperature dust collector of the dedusting and desulfurization unit to remove dust, and then passes through the dry desulfurization device of the dedusting and desulfurization unit to remove hydrogen sulfide; the circulating flue gas from the second waste heat boiler has a carbon dioxide concentration of 95.0 v % or higher, a temperature of 250-300° C., and a pressure of 0.1-0.2 MPa, and the circulating flue gas after dust removal and desulphurization has a carbon dioxide concentration of 95.0 v % or higher, a temperature of 250-300° C., and a pressure of 0.05-0.10 MPa, a portion of which is pressurized to 2.5-3.0 MPa and fed into the methanation unit, and the rest is cooled down and sold externally as the carbon dioxide products.

In some embodiments, in the initial stage, the glass furnace adopts the natural gas as the fuel and the air as the combustion aid, and after the flue gas is produced, the carbon-based enriched oxygen mixed by the flue gas, the oxygen produced by the air separation unit, and the oxygen produced by the water electrolysis hydrogen production unit gradually replaces the air and is used as the combustion aid, and after 3-5 hours of cyclic enrichment, the carbon dioxide concentration of the circulating flue gas reaches the equilibrium, with a carbon dioxide concentration of 95.0 v % or higher, the methanation unit, the reforming unit, the first waste heat boiler, the second waste heat boiler, and the dedusting and desulfurization unit are put into operation, and the system enters into the normal operation state.

### BRIEF DESCRIPTION OF THE DRAWINGS

The present disclosure is further described in terms of exemplary embodiments. These exemplary embodiments are described in detail with reference to the drawings, and the drawings are not to scale. These embodiments are non-limiting schematic embodiments, in which like reference numerals represent similar structures throughout the several views of the drawings, wherein:

FIG. 1 is a schematic diagram illustrating a structure of a system required for a zero-carbon glass furnace process according to some embodiments of the present disclosure;

FIG. 2 is a schematic diagram illustrating another structure of the system required for the zero-carbon glass furnace process according to some embodiments of the present disclosure.

### DETAILED DESCRIPTION

In order to make the purpose, technical solutions and advantages of the embodiments of the present disclosure clearer, the technical solutions in the embodiments of the present disclosure will be clearly and completely described below in conjunction with the accompanying drawings in the embodiments of the present disclosure, and it is clear that the embodiments described is a part of the embodiments of the present disclosure, and not all of the embodiments. Accordingly, the following detailed description of the embodiments of the present disclosure provided in the accompanying drawings is not intended to limit the scope of the claimed protection, but only indicates selected embodiments of the present disclosure. Based on the embodiments in the present disclosure, all other embodiments obtained by a person skilled in the art without creative labor are within the protection scope of the present disclosure.

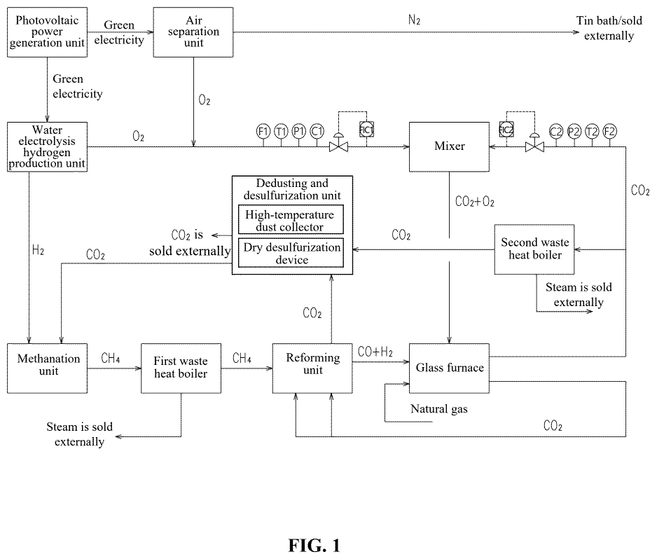

FIG. 1 is a schematic diagram illustrating a structure of a system required for a zero-carbon glass furnace process according to some embodiments of the present disclosure.

Embodiments of the present disclosure provide a zero-carbon glass furnace process (hereinafter referred to as “the process”), and the system required for the process (hereinafter referred to as “the system”) is shown in FIG. 1, including a photovoltaic power generation unit, an air separation unit, a water electrolysis hydrogen production unit, a mixer, a methanation unit, a first waste heat boiler, a reforming unit, a glass furnace, a second waste heat boiler, and a dedusting and desulfurization unit.

The photovoltaic power generation unit is configured to produce green electricity to supply electricity to the air separation unit and the water electrolysis hydrogen production unit. The main principle of photovoltaic power generation is the photoelectric effect of semiconductors. When irradiated by electromagnetic waves with a frequency higher than a specific frequency (which is called a threshold frequency), electrons within certain substances absorb energy and escape to form an electric current, that is, photo-generated electricity. The photovoltaic power generation has the advantages of safety, reliability, no noise, no polluting emission, absolute cleanliness, and no harm.

The air separation unit is configured to produce oxygen and nitrogen using the green electricity supplied by the photovoltaic power generation unit as electrical energy. The air separation unit produces oxygen and nitrogen by following operations. The air is first compressed, cooled, and liquefied, and then gas-liquid contact on the distillation tower plate is conducted for mass and heat exchange using the difference in the boiling points of the oxygen and nitrogen components, the high-boiling-point oxygen component constantly condenses into liquid from the vapor, and low-boiling-point nitrogen component constantly transfers to the vapor, so that the nitrogen content in the rising vapor constantly increases, and the oxygen content in the downward-flowing liquid becomes increasingly high, thereby separating oxygen and nitrogen.

In some embodiments, the oxygen produced by the air separation unit has a purity of 99.6 v % or higher, a normal temperature, and a pressure of 0.2-0.3 MPa, and the nitrogen produced by the air separation unit has a purity of 99.9 v % or higher, a normal temperature, and a pressure of 0.2-0.3 MPa. Since the air separation unit adopts the green electricity produced by the photovoltaic power generation unit as the electrical energy, the produced oxygen is green oxygen, and the produced nitrogen is green nitrogen. In some embodiments, the oxygen produced by the air separation unit is fed into the mixer, and mixed with the oxygen from the water electrolysis hydrogen production unit and circulating flue gas from the glass furnace to form carbon-based enriched oxygen for use in the glass furnace to aid in combustion, and a carbon dioxide concentration of the circulating flue gas is 95.0 v % or higher. In some embodiments, a portion of nitrogen produced by the air separation unit is depressurized to 0.05 MPa and then fed into the tin bath of the glass furnace as a protective gas, and the rest is sold externally.

The water electrolysis hydrogen production unit, powered by the green electricity supplied by the photovoltaic power generation unit, is configured to produce hydrogen and oxygen. Hydrogen production by water electrolysis is one of the convenient methods of producing hydrogen, in which direct current is passed through an electrolyzer filled with electrolyte, and water molecules react electrochemically on electrodes to decompose into hydrogen and oxygen.

In some embodiments, the hydrogen produced by the water electrolysis hydrogen production unit has a purity of 99.8 v % or higher, a temperature of 90-100° C., and a pressure of 2.5-3.0 MPa, and the oxygen produced by the water electrolysis hydrogen production unit has a purity of 99.0 v % or higher, a temperature of 90-100° C., and a pressure of 2.5-3.0 MPa. Since the water electrolysis hydrogen production unit uses the green electricity produced by the photovoltaic power generation unit as the electrical energy, the produced hydrogen is green hydrogen, and the produced oxygen is green oxygen. The hydrogen produced by the water electrolysis hydrogen production unit is fed into the methanation unit for the methanation reaction. In some embodiments, the oxygen produced by the water electrolysis hydrogen production unit is depressurized to 0.2-0.3 MPa and fed into the mixer, and is mixed with the oxygen from the air separation unit and the circulating flue gas from the glass furnace to form the carbon-based enriched oxygen as a combustion aid for use in the glass furnace to aid in combustion, and the circulating flue gas has a carbon dioxide concentration of 95.0 v % or higher.

Hydrogen production by water electrolysis reaction equation is 2H₂O→2H₂+O₂.

The mixer is configured to mix oxygen from the air separation unit, oxygen from the water electrolysis hydrogen production unit, and the circulating flue gas from the glass furnace to form the carbon-based enriched oxygen as the combustion aid for use in the glass furnace to aid in combustion.

In some embodiments, the oxygen from the air separation unit has a purity of 99.6 v % or higher, a normal temperature, and a pressure of 0.2-0.3 MPa. In some embodiments, the oxygen from the water electrolysis hydrogen production unit has a purity of 99.0 v % or higher, a temperature of 90-100° C., and a pressure of 0.2-0.3 MPa. In some embodiments, the circulating flue gas from the glass furnace has a carbon dioxide concentration of 95.0 v % or higher, a temperature of 550-650° C., and a pressure of 0.15-0.25 MPa. In some embodiments, the oxygen from the air separation unit, the oxygen from the water electrolysis hydrogen production unit, and the circulating flue gas from the glass furnace are adjusted through corresponding flow regulating valve (or referred to flow indicator controller), respectively and mixed to form the carbon-based enriched oxygen with an oxygen content of 21-30 v %, a temperature of 460-480° C., and a pressure of 0.05-0.10 MPa, and used as the combustion aid of the glass furnace. In some embodiments, the oxygen content may be adjusted according to the different requirements of glass furnaces.

The methanation unit is configured to methanize hydrogen from the water electrolysis hydrogen production unit and circulating flue gas from the dedusting and desulfurization unit.

In some embodiments, the hydrogen from the water electrolysis hydrogen production unit has a purity of 99.8 v % or higher, a temperature of 90-100° C., and a pressure of 2.5-3.0 MPa. In some embodiments, the circulating flue gas from the dedusting and desulfurization unit has a carbon dioxide concentration of 95.0 v % or higher, a temperature of 250-300° C., and a pressure of 2.5-3.0 MPa. In some embodiments, the hydrogen and the carbon dioxide are fed into the methanation unit at a volume ratio of H₂ and CO₂ of 4:1, and under an action of a nickel-based methanation catalyst, the hydrogen and the carbon dioxide are catalyzed to generate methane containing saturated water vapor, while releasing a large amount of heat, the methane has a purity of 94.0 v % or higher on a dry basis, a temperature of 390-410° C., and a pressure of 2.5-3.0 MPa, and the methane containing saturated water vapor is fed into a first waste heat boiler. Exemplarily, the nickel-based catalyst may be Ni/Al₂O₃, Ni/SiO₂, or the like.

Methanation reaction equation is 4H₂+CO₂→CH₄+2H₂O.

The first waste heat boiler is configured to recover waste heat of methane containing saturated water vapor from the methanation unit and by-produce steam.

In some embodiments, the methane containing saturated water vapor from the methanation unit has a temperature of 390-410° C., the temperature is lowered to 240-260° C. after waste heat recovery from the first waste heat boiler and steam with a pressure of 1.5 MPa is by-produced for external sale. Since a suitable pressure for the reforming reaction is 0.5-1.0 MPa, in some embodiments, the methane containing saturated water vapor after waste heat recovery is depressurized to 0.5-1.0 MPa and then fed into the reforming unit.

The reforming unit is configured to subject the methane containing saturated water vapor from the first waste heat boiler and a portion of the circulating flue gas from the glass furnace to a methane reforming reaction.

In some embodiments, the methane containing saturated water vapor from the first waste heat boiler has a methane purity of 94.0 v % or higher on a dry basis, a temperature of 240-260° C., and a pressure of 0.5-1.0 MPa. In some embodiments, the circulating flue gas from the glass furnace has a carbon dioxide concentration of 95.0 v % or higher, a temperature of 1300-1400° C., and a pressure of 0.5-1.0 MPa. In some embodiments, the circulating flue gas from the glass furnace is divided into two ways to enter the reforming unit, one way enters the heat storage grid on the one side of the reforming unit, providing heat for the heat storage grid on the one side of the reforming unit to provide heat for the raw gas of the reforming unit in the next round, after heat exchange, the temperature of the circulating flue gas is reduced to 250-300° C., and the pressure of the circulating flue gas is reduced to 0.1-0.2 MPa and then fed into the dedusting and desulfurization unit; the other way and the methane containing saturated water vapor from the first waste heat boiler are fed into the heat storage grid on the other side of the reforming unit at a volume ratio of CH₄ and CO₂ of 2:1, the heat storage grid on the other side of the reforming unit is heated by the circulating flue gas of the previous round, the two heat storage grids work in rotation, and the raw gas fed into the reforming unit is heated to 900-1000° C. by the heat storage grid on the other side of the reforming unit, under a pressure of 0.5-1.0 MPa and the nickel-based catalyst, the methane, the carbon dioxide, and the water vapor undergo the methane reforming reaction in the reaction bed of the reforming unit to produce the carbon monoxide and the hydrogen, with a volume ratio of CO and H₂ of 3:5, an outlet temperature of 700-800° C., and a pressure of 0.5-1.0 MPa, and the carbon monoxide and the hydrogen are fed into the glass furnace as the fuel.

Reforming reaction equation is CH₄+H₂O→CO+3H₂ and CH₄+CO₂→2CO+2H₂.

The glass furnace is configured to produce the molten glass using carbon monoxide and hydrogen from the reforming unit as the fuel (natural gas is used as the fuel in an initial stage of production) and the carbon-based enriched oxygen from the mixer as the combustion aid.

In some embodiments, the carbon monoxide and the hydrogen from the reforming unit have a volume ratio of CO and H₂ of about 3:5, a temperature of 700-800° C., and a pressure of 0.5-1.0 MPa. In some embodiments, the carbon-based enriched oxygen from the mixer has an oxygen content of 21-30 v %, a temperature of 460-480° C., and a pressure of 0.05-0.10 MPa; the carbon-based enriched oxygen from the mixer first passes through the heat storage grid of the one side of the glass furnace, the heat storage grid is heated by the previous round of circulating flue gas, the two sides of the heat storage grids work in rotation, and the carbon-based enriched oxygen from the mixer is heated to 700-800° C. and then fed into the glass furnace. In some embodiments, the fuel and the combustion aid have a volume ratio of about 1:2. The fuel refers to the carbon monoxide and the hydrogen from the reforming unit, and the combustion aid refers to the carbon-based enriched oxygen from the mixer. In the glass furnace, under a combustion of the carbon-based enriched oxygen, the carbon monoxide and the hydrogen undergo a combustion reaction, providing heat for the production of the molten glass in the glass furnace.

Carbon-based enriched oxygen combustion reaction equation is 2CO+O₂+CO₂→3CO₂ and 2H₂+O₂+CO₂→2H₂O+CO₂.

In some embodiments, after the flue gas of the glass furnace is circularly enriched 3-5 hours, the carbon dioxide concentration reaches the equilibrium, with the carbon dioxide concentration of 95.0 v % or higher, a temperature of 1300-1400° C., and a pressure of 0.01-0.05 MPa. The concentration reaching the equilibrium refers to a concentration fluctuation of less than ±0.5% for 1 hour of continuous monitoring. In some embodiments, the circulating flue gas is divided into two ways, one way passes through the heat storage grid on the other side of the glass furnace, providing heat for the heat storage grid on the other side of the glass furnace to provide heat for the carbon-based enriched oxygen from the mixer in the next round, after heat exchange, the circulating flue gas of this way is cooled down to 550-650° C. and pressurized to 0.15-0.25 MPa, and the circulating flue gas is fed into the mixer and the second waste heat boiler; the other way does not pass through the heat storage grid of the glass furnace, with a temperature of 1300-1400° C., is pressurized to 0.5-1.0 MPa, and fed into the reforming unit.

The second waste heat boiler is configured to recover waste heat of the circulating flue gas from the glass furnace and by-produce steam.

In some embodiments, the circulating flue gas from the glass furnace has a carbon dioxide concentration of 95.0 v % or higher, a temperature of 550-650° C., and a pressure of 0.15-0.25 MPa, after waste heat recovery by the second waste heat boiler, the temperature is reduced to 250-300° C., and the pressure is 0.1-0.2 MPa, and the steam with a pressure of 1.5-4.0 MPa is by-produced for external sale. In some embodiments, the circulating flue gas after waste heat recovery is fed into the dedusting and desulfurization unit.

The dedusting and desulfurization unit is configured to dedust and desulfurize circulating flue gas from the second waste heat boiler.

In some embodiments, the dedusting and desulfurization unit includes a high-temperature dust collector and a dry desulfurization device. The high-temperature dust collector adopts cyclone separating and dust removing, and the dry desulfurization device adopts zinc oxide desulfurization. In some embodiments, the circulating flue gas first passes through the high-temperature dust collector of the dedusting and desulfurization unit to remove dust, and then passes through the dry desulfurization device of the dedusting and desulfurization unit to remove hydrogen sulfide.

In some embodiments, the circulating flue gas from the second waste heat boiler has a carbon dioxide concentration of 95.0 v % or higher, a temperature of 250-300° C., and a pressure of 0.1-0.2 MPa. The circulating flue gas after dust removal and desulphurization has a carbon dioxide concentration of 95.0 v % or higher, a temperature of 250-300° C., and a pressure of 0.05-0.10 MPa, a portion of which is pressurized to 2.5-3.0 MPa and fed into the methanation unit, and the rest is cooled down and sold externally as the carbon dioxide products.

Dry desulfurization reaction equation is ZnO+H₂S→ZnS+H₂O.

In some embodiments, as shown in FIG. 1, the photovoltaic power generation unit is connected to the air separation unit and the water electrolysis hydrogen production unit, respectively; a nitrogen outlet of the air separation unit is connected to a tin bath of the glass furnace and to a nitrogen product storage tank, respectively, and a pressure reducing valve is provided on a connecting pipe between the nitrogen outlet of the air separation unit and the tin bath of the glass furnace; an oxygen outlet of the air separation unit and an oxygen outlet of the water electrolysis hydrogen production unit are both connected to the mixer, a pressure reducing valve is provided on a connecting pipe between the oxygen outlet of the water electrolysis hydrogen production unit and the mixer, a connecting pipe between the oxygen outlet of the air separation unit, the oxygen outlet of the water electrolysis hydrogen production unit, and the mixer is provided with a flow detector F1, a temperature detector T1, a pressure detector P1, an oxygen purity detector C1, and a flow regulating valve FIC1; a hydrogen outlet of the water electrolysis hydrogen production unit is connected to the methanation unit; a carbon-based enriched oxygen outlet of the mixer is switchably connected to a heat storage grid A/B of the glass furnace and then connected to the glass furnace; a methane outlet of the methanation unit is connected to the first waste heat boiler; a methane outlet of the first waste heat boiler is switchably connected to a heat storage grid A/B of the reforming unit and then connected to the reforming unit, a pressure reducing valve is provided on a connecting pipe between the methane outlet of the first waste heat boiler and the heat storage grid A/B of the reforming unit, and a steam outlet of the first waste heat boiler is connected to a steam utilizer; a carbon monoxide and hydrogen outlet of the reforming unit is connected to the glass furnace; one of circulating flue gas outlets of the glass furnace is switchably connected to the heat storage grid B/A of the glass furnace and then connected to the second waste heat boiler and the mixer, one of the circulating flue gas outlets of the glass furnace is switchably connected to the heat storage grid A/B of the reforming unit and then connected to the reforming unit, and one of the circulating flue gas outlets of the glass furnace is switchably connected to a heat storage grid B/A of the reforming unit and then connected to the dedusting and desulfurization unit, respectively, a pressure booster is provided on a connecting pipe between the heat storage grid B/A of the glass furnace, the second waste heat boiler, and the mixer, a connecting pipe between the heat storage grid B/A of the glass furnace and the mixer is provided with a flow detector F2, a temperature detector T2, a pressure detector P2, a carbon dioxide purity detector C2, and a flow regulating valve FIC2, a pressure booster is provided on the connecting pipe between the glass furnace, the heat storage grid A/B of the reforming unit, and the heat storage grid B/A of the reforming unit, and a pressure reducing valve is provided on a connecting pipe between the heat storage grid B/A of the reforming unit and the dedusting and desulfurization unit; a circulating flue gas outlet of the second waste heat boiler is connected to the dedusting and desulfurization unit; circulating flue gas outlets of the dedusting and desulfurization unit are connected to the methanation unit and a carbon dioxide product storage tank, respectively, and a pressure booster is provided on a connecting pipe between one of the circulating flue gas outlets of the dedusting and desulfurization unit and the methanation unit.

The heat storage grid refers to a structure that exchanges heat through heat storage and heat release of medium, which may be in a shape of a grid body. In some embodiments, the previously described switchable connection of the respective device to the heat storage grid A/B or the heat storage grid B/A may refer to a connection via a switching valve (not shown in the figures) that switches the connected heat storage grid after every heat exchange cycle. The heat exchange cycle refers to a complete working period experienced by one side of the heat storage grid from “heat absorption” to “heat release”. Exemplarily, a duration of each heat exchange cycle may be set to a preset duration, and the switching valve switches every preset duration, e.g., switching the connection from the heat storage grid A to the heat storage grid B, and the switching the connection from heat storage grid B to the heat storage grid B. More information regarding the heat exchange cycle and the switching process may be found in the following section of the present disclosure. The preset duration may be preset by a process expert.

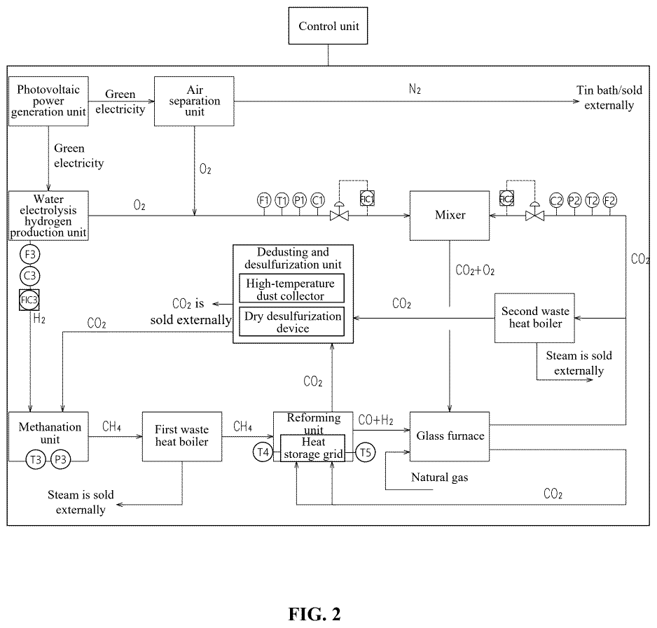

FIG. 2 is a schematic diagram illustrating another structure of the system required for the zero-carbon glass furnace process according to some embodiments of the present disclosure.

In some embodiments, as shown in FIG. 2, the system required for the process further includes the following devices: a flow detector F3, a hydrogen purity detector C3, and a flow regulating valve FIC3 provided at a hydrogen outlet of the water electrolysis hydrogen production unit; a temperature detector T3 and a pressure detector P3 provided in the methanation unit; a bypass cooling gas valve provided at an inlet of the methanation unit; a temperature detector T4 and a temperature detector T5 provided at an inlet and at an outlet of the heat storage grid of the reforming unit, respectively; and a control unit.

In some embodiments, the flow detector F3 and the hydrogen purity detector C3 are configured to obtain an instantaneous total flow rate and a purity of the hydrogen, respectively, and the flow regulating valve FIC3 is configured to regulate a flow rate of the hydrogen.

In some embodiments, the temperature detector T3 is configured to monitor a temperature within the methanation unit in real time, and the pressure detector P3 is configured to monitor a pressure within the methanation unit in real time.

In some embodiments, the bypass cooling gas valve is configured to introduce a cooling gas when the methanation unit is overheating. In some embodiments, overheating may refer to a temperature within the methanation unit exceeding a temperature threshold, which may be determined based on empirical presets. The cooling gas refers to a gas used to lower the temperature in the methanation unit. For example, the cooling gas may be high purity nitrogen supplied by an air separation unit, or other gases that do not interfere with the main reaction of the methanization.

In some embodiments, the temperature detector T4 on the inlet of the heat storage grid and the temperature detector T5 on the outlet of the heat storage grid are configured to monitor a temperature difference of the gas entering and exiting the heat storage grid in real time, to determine whether the heat storage grid is available to provide heat to the raw gas of the reforming unit, so as to switch the entry path of the circulating flue gas in a timely manner. More detailed description regarding the determination manner and the switching manner may be found in the following section of the present disclosure.

The control unit refers to a device configured to collect process parameters and control the operation state of each device. In some embodiments, the control unit may be an industrial automation control system, such as a programmable logic controller (PLC), a distributed control system (DCS), an embedded controller, or the like. In some embodiments, the control unit may be configured to perform zero-carbon glass furnace process operations. In some embodiments, the control unit may be disposed at any feasible location within the system required for the process. For example, the control unit may be disposed independently with respect to other devices within the system required for the process and arranged around the methanation unit, the reforming unit, or a glass furnace. The control unit may be connected to the respective detection devices and actuating components by means of a signal line or bus.

In some embodiments, the zero-carbon glass furnace process includes the following operations.
(1) The green electricity is produced by the photovoltaic power generation unit to supply the electricity for the air separation unit and the water electrolysis hydrogen production unit; oxygen and nitrogen are produced by the air separation unit with the green electricity provided by the photovoltaic power generation unit as the electrical energy, then the oxygen is fed into the mixer, a portion of the nitrogen is depressurized and then fed into the tin bath of the glass furnace as protective gas, and the rest is sold outside; hydrogen and oxygen are produced by the water electrolysis hydrogen production unit with the green electricity provided by the photovoltaic power generation unit as the electrical energy, then the hydrogen is fed into the methanation unit, and the oxygen is fed into the mixer after depressurization.
(2) The oxygen from the air separation unit, the oxygen from the water electrolysis hydrogen production unit, and the circulating flue gas from the glass furnace are mixed into the carbon-based enriched oxygen by the mixer, and the carbon-based enriched oxygen is used as the combustion aid for the glass furnace.
(3) The hydrogen from the water electrolysis hydrogen production unit and the circulating flue gas from the dedusting and desulfurization unit are methanized by the methanation unit to produce the methane containing saturated water vapor, and feeding the methane containing saturated water vapor into the first waste heat boiler.
(4) The waste heat of the methane containing saturated water vapor from the methanation unit is recovered and the steam is by-produced by the first waste heat boiler; the steam is sold externally, and depressurized methane containing saturated water vapor after waste heat recovery is fed into the reforming unit.
(5) The methane containing saturated water vapor from the first waste heat boiler and the portion of the circulating flue gas from the glass furnace are subjected to the methane reforming reaction by the reforming unit; the circulating flue gas from the glass furnace is fed into the reforming unit in two ways, one way is fed into a heat storage grid of one side of the reforming unit, providing heat for the heat storage grid of the one side of the reforming unit to provide heat for raw gas of the reforming unit in a next round, and after heat exchange, the circulating flue gas is depressurized and fed into the dedusting and desulfurization unit; the other way is fed into a heat storage grid on the other side of the reforming unit along with the methane containing saturated water vapor from the first waste heat boiler, the heat storage grid on the other side of the reforming unit is already heated by the circulating flue gas of a previous round, two sides of the heat storage grids work in rotation, and the raw gas fed into the reforming unit is heated by heat of the heat storage grid on the other side of the reforming unit, in a reaction bed of the reforming unit, under a certain pressure and a certain catalyst, methane, carbon dioxide, and water vapor are subjected to the methane reforming reaction to produce the carbon monoxide and the hydrogen, and the carbon monoxide and the hydrogen are fed into the glass furnace as the fuel.
(6) The molten glass is produced by the glass furnace using the carbon monoxide and the hydrogen from the reforming unit as the fuel and the carbon-based enriched oxygen from the mixer as the combustion aid; the carbon-based enriched oxygen from the mixer first passes through a heat storage grid on one side of the glass furnace, the heat storage grid on the one side of the glass furnace is already heated by the previous round of the circulating flue gas, two sides of the heat storage grids work in rotation, and the carbon-based enriched oxygen from the mixer is heated to a certain temperature and then fed into the glass furnace. After the flue gas of the glass furnace is circularly enriched for a certain period of time, a carbon dioxide concentration reaches an equilibrium; the circulating flue gas is divided into two ways, one way passes through a heat storage grid on the other side of the glass furnace, providing heat for the heat storage grid on the other side of the glass furnace to provide heat for the carbon-based enriched oxygen from the mixer in the next round, and after heat exchange, the circulating flue gas of this way is pressurized and fed into the mixer and the second waste heat boiler; the other way does not pass through the heat storage grid of the glass furnace, and is pressurized and fed into the reforming unit.
(7) The waste heat of the circulating flue gas from the glass furnace is recovered and the steam is by-produced by the second waste heat boiler; the steam is sold externally, and the circulating flue gas after waste heat recovery is fed into the dedusting and desulfurization unit.
(8) The circulating flue gas from the second waste heat boiler is dedusted and desulfurized by the dedusting and desulfurization unit; a portion of the circulating flue gas is pressurized after dedusting and desulfurization and pressurized circulating flue gas is fed into the methanation unit, and the rest is sold externally after cooled down as carbon dioxide products.

In an initial stage, the glass furnace uses natural gas as the fuel and air as the combustion aid, and after flue gas is produced, the carbon-based enriched oxygen mixed by the flue gas, the oxygen produced by the air separation unit, and the oxygen produced by the water electrolysis hydrogen production unit gradually replaces the air and is used as the combustion aid, after a certain period of cyclic enrichment, the carbon dioxide concentration of the circulating flue gas reaches the equilibrium, and the methanation unit, the reforming unit, the first waste heat boiler, the second waste heat boiler, and the dedusting and desulfurization unit are put into operation, and the system enters into a normal operation state. The initial stage refers to a stage when the system required for the process is just beginning to be put into operation. The normal operation state refers to a state in which the system required by the process circulates the above operations (1)-(8).

In some embodiments, operation (1) may include following contents. The green electricity is produced by the photovoltaic power generation unit to supply the electricity for the air separation unit and the water electrolysis hydrogen production unit. The air separation unit produces oxygen and nitrogen with the green electricity provided by the photovoltaic power generation unit as the electrical energy, the oxygen has a purity of 99.6 v % or higher, a normal temperature, and a pressure of 0.2-0.3 MPa, and the oxygen is fed into the mixer; and the nitrogen has a purity of 99.9 v % or higher, a normal temperature, and a pressure of 0.2-0.3 MPa, the portion of the nitrogen is depressurized to 0.05 MPa and fed into the tin bath of the glass furnace as the protective gas, and the rest is sold externally. The water electrolysis hydrogen production unit produces oxygen and nitrogen with the green electricity provided by the photovoltaic power generation unit as the electrical energy, the hydrogen has a purity of 99.8 v % or higher, a temperature of 90-100° C., and a pressure of 2.5-3.0 MPa, and the hydrogen is fed into the methanation unit; and the oxygen has a purity of 99.0 v % or higher, a temperature of 90-100° C., and a pressure of 2.5-3.0 MPa, and the oxygen is depressurized to 0.2-0.3 MPa and fed into the mixer.

In some embodiments, operation (2) may include following contents. The mixer mixes the oxygen from the air separation unit, the oxygen from the water electrolysis hydrogen production unit, and the circulating flue gas from the glass furnace into the carbon-based enriched oxygen as the combustion aid for the glass furnace. The oxygen from the air separation unit has a purity of 99.6 v % or higher, a normal temperature, and a pressure of 0.2-0.3 MPa. The oxygen from the water electrolysis hydrogen production unit has a purity of 99.0 v % or higher, a temperature of 90-100° C., and a pressure of 0.2-0.3 MPa. The circulating flue gas from the glass furnace has a carbon dioxide concentration of 95.0 v % or higher, a temperature of 550-650° C., and a pressure of 0.15-0.25 MPa. The oxygen from the air separation unit, the oxygen from the water electrolysis hydrogen production unit, and the circulating flue gas from the glass furnace are each regulated in flow rate via flow regulating valves, the carbon-based enriched oxygen has an oxygen content of 21-30 v %, a temperature of 460-480° C., and a pressure of 0.05-0.10 MPa, and the oxygen content is adjusted according to different requirements of the glass furnace.

In some embodiments, operation (3) may include following contents. The hydrogen from the water electrolysis hydrogen production unit and the circulating flue gas from the dedusting and desulfurization unit are methanized by the methanation unit. The hydrogen from the water electrolysis hydrogen production unit has a purity of 99.8 v % or higher, a temperature of 90-100° C., and a pressure of 2.5-3.0 MPa. The circulating flue gas from the dedusting and desulfurization unit has a carbon dioxide concentration of 95.0 v % or higher, a temperature of 250-300° C., and a pressure of 2.5-3.0 MPa. The hydrogen and the circulating flue gas are fed into the methanation unit at a volume ratio of H₂ and CO₂ of 4:1, under an action of a nickel-based methanation catalyst, the hydrogen and the carbon dioxide are catalyzed to generate methane, while releasing a large amount of heat, the methane has a purity of 94.0 v % or higher on a dry basis, a temperature of 390-410° C., and a pressure of 2.5-3.0 MPa, and the methane containing saturated water vapor is fed into the first waste heat boiler.

In some embodiments, operation (3) further includes following contents. The control unit obtains a hydrogen supply volume and a carbon dioxide supply volume, determines a current volume ratio of the hydrogen supply volume to the carbon dioxide supply volume based on the hydrogen supply volume and the carbon dioxide supply volume; and in response to determining that the volume ratio of the hydrogen supply volume to the carbon dioxide supply volume deviates from a target ratio, adjusts the flow regulating valve FIC3 for the hydrogen and/or the flow regulating valve FIC2 for the carbon dioxide until the volume ratio of the hydrogen supply volume to the carbon dioxide supply volume returns to the target ratio.

The hydrogen supply volume refers to a flow rate of hydrogen actually involved in the methanation reaction. In some embodiments, the control unit may obtain the hydrogen supply volume in a plurality of ways. For example, the control unit may determine a product of a total flow rate of the hydrogen branch and the hydrogen purity as the hydrogen supply volume. The total flow rate of the hydrogen branch refers to a total volume of gas passing through the hydrogen supply branch per unit of time. In some embodiments, the control unit may determine the total flow rate of the hydrogen branch by the flow detector F3. The hydrogen purity refers to a volume fraction of the hydrogen in the gas. In some embodiments, the control unit may determine the hydrogen purity by the hydrogen purity detector C3.

The carbon dioxide supply volume refers to a flow rate of carbon dioxide actually involved in the methanation reaction. In some embodiments, the control unit may obtain the carbon dioxide supply volume in a plurality of ways. For example, the control unit may determine a product of a total flow rate of the carbon dioxide branch and a carbon dioxide purity as the carbon dioxide supply volume. The total flow rate of the carbon dioxide branch refers to a total volume of gas passing through the carbon dioxide branch per unit of time. The control unit may determine the total flow rate of the carbon dioxide branch by the flow detector F2. The carbon dioxide purity refers to a volume fraction of carbon dioxide in the gas. In some embodiments, the control unit may determine the carbon dioxide purity by the carbon dioxide purity detector C2.

In some embodiments, the control unit may determine a ratio of the hydrogen supply volume and the carbon dioxide supply volume as a current volume ratio of the hydrogen supply volume to the carbon dioxide supply volume based on the hydrogen supply volume and the carbon dioxide supply volume.

In some embodiments, there are cases where the volume ratio of the hydrogen supply volume to the carbon dioxide supply volume deviates, or does not deviate, from the target ratio. In some embodiments, in response to determining that the volume ratio of the hydrogen supply volume to the carbon dioxide supply volume deviates from the target ratio, the control unit may adjust the flow regulating valve FIC3 for the hydrogen and/or the flow regulating valve FIC2 for the carbon dioxide until the volume ratio of the hydrogen supply volume to the carbon dioxide supply volume returns to the target ratio. The target ratio is 4:1.

A deviation degree refers to a degree to which the volume ratio of the hydrogen supply volume to the carbon dioxide supply volume differs from the target ratio. In some embodiments, the control unit may calculate a percentage difference between the volume ratio of the hydrogen supply volume to the carbon dioxide supply volume and the target ratio, determine the percentage difference as the deviation degree, and according to the deviation degree, perform the corresponding adjustment to return the volume ratio of the hydrogen supply volume to the carbon dioxide supply volume to the target ratio. For example, in response to determining that the deviation degree is less than a first deviation threshold, the control unit may adjust the flow regulating valve FIC3 and/or the flow regulating valve FIC2 using a micro-step adjustment. As another example, in response to determining that the deviation degree is greater than the first deviation threshold and less than the second deviation threshold, the control unit may adjust the flow regulating valve FIC3 and/or the flow regulating valve FIC2 using a proportional adjustment. As another example, in response to determining that the deviation degree is greater than a second deviation threshold, the control unit may adjust the flow regulating valve FIC3 and/or the flow regulating valve FIC2 using a fast adjustment. The first deviation threshold may be determined based on an empirical preset, e.g., 2%. The second deviation threshold is greater than the first deviation threshold, and the second deviation threshold may be determined based on an empirical preset, e.g., 5%.

The above three adjustment modes gradually increase the magnitude of regulation of the flow regulating valve based on an increase of the deviation degree. The micro-step adjustment refers to a small-amplitude adjustment of the flow regulating valve, e.g., a single adjustment of an opening magnitude of the flow regulating valve is controlled to be within 0.5%-1%. The proportional adjustment refers to an adjustment of the flow regulating valve proportionally according to the magnitude of the deviation degree, with the change in the valve opening being positively correlated with the deviation degree of the volume ratio. The fast adjustment refers to a large-scale adjustment of the flow regulating valve, e.g., a single adjustment of the opening magnitude of the flow regulating valve is controlled to be within 5-10%.

In some embodiments of the present disclosure, the optimization of the process control of the volume ratio of the methanation reaction is achieved by monitoring and adjusting the hydrogen supply volume and the carbon dioxide supply volume in real time, which can effectively guarantee the “zero-carbon” operation goal.

In some embodiments, operation (3) further includes following contents. The control unit obtains a temperature of the methanization reaction; and in response to determining that the temperature of the methanization reaction exceeds an upper temperature limit, open the bypass cooling gas valve to introduce the cooling gas to reduce the temperature to an allowable range.

In some embodiments, the control unit may obtain the temperature of the methanation reaction by the temperature detector T3. In some embodiments, there are cases where the temperature of the methanation reaction exceeds, or does not exceed, an upper temperature limit. In some embodiments, in response to determining that the temperature of the methanation reaction exceeds the upper temperature limit, the control unit may open the bypass cooling gas valve to introduce the cooling gas to reduce the temperature to the allowable range. The upper temperature limit refers to 410° C. and the allowable range refers to 390-410° C. When the temperature drops back to the allowable range, the control unit may close the bypass cooling gas valve and stop introducing the cooling gas.

In some embodiments of the present disclosure, by monitoring the temperature of the methanation reaction in real time and controlling the input of the cooling gas via the bypass cooling gas valve, it is possible to prevent the catalyst failure triggered by overheating of the methanation unit, to enhance the operational stability of the methanation unit.

In some embodiments, operation (3) further includes following contents. The control unit determines a target opening of the flow regulating valve FIC3 for the hydrogen and a target opening of the flow regulating valve FIC2 for the carbon dioxide using an opening determination model based on a deviation degree sequence of the volume ratio of the hydrogen supply volume to the carbon dioxide supply volume.

The deviation degree sequence is a sequence including a plurality of time points and the corresponding deviation degrees. For example, the deviation degree sequence may be expressed as {(time point A, deviation degree A), (time point B, deviation degree B), (time point C, deviation degree C.)}. More descriptions regarding the deviation degree may be found above.

The opening determination model refers to a model used to determine the target opening of the flow regulating valve. In some embodiments, the opening determination model may be a machine learning model. For example, the opening determination model may be a Neural Network (NN), a Recurrent Neural Network (RNN), etc. In some embodiments, an input to the opening determination model may include the deviation degree sequence of the volume ratio of the hydrogen supply volume to the carbon dioxide supply volume and environmental information, and an output of the opening determination model may include a target opening.

The environmental information refers to data related to an environment of the methanation unit. For example, the environmental information may include a temperature, a pressure, or the like in the methanation unit. In some embodiments, the control unit may obtain the environmental information in real time through devices such as the temperature detector T3, the pressure detector P3, or the like.

The target opening refers to a valve opening that enables a volume ratio of the hydrogen supply volume to the carbon dioxide supply volume to return to a target range. The target range may be determined based on a manual preset, e.g., a range of ±1% from the target ratio. In some embodiments, the target opening may include a target opening of the flow regulating valve FIC3 and a target opening of the flow regulating valve FIC2.

In some embodiments, the control unit may construct the opening determination model by training with a large number of training samples and training labels corresponding to the training samples. In some embodiments, the control unit may obtain a training dataset, the training dataset including a plurality of training samples and training labels corresponding to the training samples, and the control unit may perform a plurality of rounds of iterations, at least one round of which includes: selecting one or more training samples from the training dataset, inputting the training samples with the training labels into an initial opening determination model to output a result of the initial opening determination model, substituting the training labels of the one or more training samples and the results of the initial opening determination model into a preset loss function, and iteratively updating parameters of the initial opening determination model based on the calculated value of the preset loss function by gradient descent or other manners. The training is completed when a preset condition is met, and the trained opening determination model is obtained. The preset condition may be that the loss function converges, a count of rounds of iterations reaches a threshold, etc.

In some embodiments, the control unit may determine a plurality of preferred regulating samples from the historical data and construct training samples and corresponding training labels based on the preferred regulating samples. In some embodiments, each of regulation records in the preferred regulating samples includes a volume ratio of the hydrogen supply volume to the carbon dioxide supply volume prior to the regulation, environmental information, and a target opening used for the regulation, and the control unit may convert the deviation degree of the volume ratio corresponding to the preferred regulation sample and the environmental information as a training sample, and the target opening of the flow regulating valve corresponding to the preferred regulation sample as a corresponding training label. The preferred regulation samples refer to regulation records in the historical data that meets a preset regulation condition. The preset regulation condition may include a time for a volume ratio of the hydrogen supply volume to the carbon dioxide supply volume to return to a target range less than a predetermined time. The preset time may be preset manually.

In some embodiments, the control unit may regulate the flow regulating valve FIC3 and the flow regulating valve FIC2 to the corresponding openings, respectively, according to the target openings.

In some embodiments of the present disclosure, by determining the target opening of the flow regulating valves using a trained machine learning model, accurate regulation of the flow regulating valves can be realized, and the efficiency and accuracy of the flow regulation can be improved.

In some embodiments, operation (4) may include following contents. The waste heat of the methane containing saturated water vapor from the methanation unit is recovered and the steam is by-produced by the first waste heat boiler. The methane containing saturated water vapor from the methanation unit has a temperature of 390-410° C., the temperature is lowered to 240-260° C. after waste heat recovery by the first waste heat boiler and steam with a pressure of 1.5 MPa is by-produced for external sale. The methane containing saturated water vapor after the waste heat recovery is depressurized to 0.5-1.0 MPa and feed into the reforming unit.

In some embodiments, operation (5) may include following contents. The methane containing saturated water vapor from the first waste heat boiler and the portion of the circulating flue gas from the glass furnace are subjected to the methane reforming reaction by the reforming unit. The methane containing saturated water vapor from the first waste heat boiler has a methane purity of 94.0 v % or higher on a dry basis, a temperature of 240-260° C., and a pressure of 0.5-1.0 MPa. The circulating flue gas from the glass furnace has a carbon dioxide concentration of 95.0 v % or higher, a temperature of 1300-1400° C., and a pressure of 0.5-1.0 MPa. The circulating flue gas from the glass furnace is divided into two ways to enter the reforming unit, one way enters the heat storage grid on the one side of the reforming unit, providing heat for the heat storage grid on the one side of the reforming unit to provide heat for the raw gas of the reforming unit in the next round, after heat exchange, the temperature of the circulating flue gas is reduced to 250-300° C., and the pressure of the circulating flue gas is reduced to 0.1-0.2 MPa and then fed into the dedusting and desulfurization unit; the other way and the methane containing saturated water vapor from the first waste heat boiler are fed to the heat storage grid on the other side of the reforming unit at a volume ratio of CH₄ and CO₂ of 2:1, the heat storage grid on the other side of the reforming unit is heated by the circulating flue gas of the previous round, the two heat storage grids work in rotation, and the raw gas fed into the reforming unit is heated to 900-1000° C. by the heat storage grid on the other side of the reforming unit, under a pressure of 0.5-1.0 MPa and a nickel-based catalyst, the methane, the carbon dioxide, and the water vapor undergo the methane reforming reaction in the reaction bed of the reforming unit to produce the carbon monoxide and the hydrogen, with a volume ratio of CO and H₂ of about 3:5, an outlet temperature of 700-800° C., and a pressure of 0.5-1.0 MPa, and the carbon monoxide and the hydrogen are fed into the glass furnace as the fuel.

In some embodiments, a duration of each heat exchange cycle may be variable, and operation (5) further includes following contents. The control unit obtains a temperature difference between the inlet temperature and the outlet temperature of the heat storage grid of the reforming unit; and in response to determining that the temperature difference decreases to be less than or equal to a preset temperature difference threshold, switches entry paths of the circulating flue gas.

In some embodiments, the control unit may determine the temperature difference between the inlet temperature and the outlet temperature of the heat storage grid of the reforming unit based on real-time data of the temperature detector T4 at the inlet of the heat storage grid of the reforming unit and the temperature detector T5 at the outlet of the heat storage grid of the reforming unit.

In some embodiments, during the heat storage process of the heat storage grid, as the heat storage grid continues to absorb heat, the heat absorption capacity of the heat storage grid decreases, and the outlet temperature of the heat storage grid continues to increase, while the inlet temperature remains relatively stable, therefore the temperature difference between the inlet temperature and the outlet temperature of the heat storage grid gradually decreases. The temperature difference reducing to a certain degree represents that the heat storage process of the heat storage grid is complete, at this time, the entry paths of the circulate flue gas should be switched, so that the heat storage grid release heat, and the other side of the heat storage grid begins to store heat to realize a rotation work of the two sides of the heat storage grids.

In some embodiments, in response to determining that the temperature difference decreases to be less than or equal to the preset temperature difference threshold, the control unit may switch the entry paths of the circulating flue gas. For example, in response to determining that the temperature difference decreases to be less than or equal to the preset temperature difference threshold, the control unit may control a switching valve of the reforming unit to switch the heat storage grids connected to the different branches, e.g., switching the heat storage grid A/B to the heat storage grid B/A, thereby switching the entry paths of the circulating flue gas. Exemplarily, in switching the heat storage grids connected to different branch paths, the control unit may first close the current circulating flue gas inlet of the heat storage grid on the current side and open the circulating flue gas inlet of the heat storage grid on the opposite side, so as to cause the heat storage grid on the opposite side to start a new round of heat storage. At the same time, the branch where the raw gas is located is switched and connected to the heat storage grid that has finished heat storage, so that the heat storage grid that has finished heat storage can be transferred to the heat release state.

In some embodiments, the preset temperature difference threshold may be determined as follows: a base value (e.g., 100° C.) is first set by a process expert based on experience, and the control unit then dynamically adjusts the preset temperature difference threshold for each heat exchange cycle based on the real-time collected heat source temperature. The heat source temperature refers to the inlet temperature of the circulating flue gas fed into the heat storage grid of the reforming unit, which may be collected in real time by the temperature detector T4 at the inlet of the heat storage grid of the reforming unit.

In some embodiments, a preset temperature difference threshold ΔT for each heat exchange cycle may be positively correlated with the real-time collected heat source temperature. For example, the preset temperature difference threshold may be determined by the following equation (1):

$$Δ T = Δ {T}_{0} + k * {T}_{1} . \qquad ( 1 )$$

Where ΔT denotes a preset temperature difference threshold, ΔT₀ denotes a preset base value, k denotes a dimensionless empirical coefficient that may be preset empirically, and T₁ denotes the heat source temperature. In some embodiments, the control unit may re-determine a preset temperature difference threshold at the beginning of each heat exchange cycle.

In some embodiments of the present disclosure, by monitoring the temperature difference between the inlet temperature and the outlet temperature of the heat storage grid, the entry paths of the circulating flue gas are switched in a timely manner to ensure that the raw gas entering the reforming unit is always preheated to a high temperature of 900-1000° C., avoiding low-temperature feeding due to late switching or insufficient heat storage due to early switching, thus ensuring the efficiency of the reforming reaction and a heat balance of the system.

In some embodiments, operation (6) may include following contents. The molten glass is produced by the glass furnace using the carbon monoxide and the hydrogen from the reforming unit as the fuel (natural gas is used as the fuel during the initial stage of production) and the carbon-based enriched oxygen from the mixer as the combustion aid. The carbon monoxide and the hydrogen from the reforming unit have a volume ratio of CO and H₂ of about 3:5, a temperature of 700-800° C., and a pressure of 0.5-1.0 MPa. The carbon-based enriched oxygen from the mixer has an oxygen content of 21-30 v %, a temperature of 460-480° C., and a pressure of 0.05-0.10 MPa; the carbon-based enriched oxygen from the mixer first passes through the heat storage grid of the one side of the glass furnace, the heat storage grid is heated by the previous round of circulating flue gas, the two sides of the heat storage grids work in rotation, and the carbon-based enriched oxygen from the mixer is heated to 700-800° C. and then fed into the glass furnace. The fuel and the combustion aid have a volume ratio of about 1:2. The fuel refers to the carbon monoxide and the hydrogen from the reforming unit, and the combustion aid refers to the carbon-based enriched oxygen from the mixer. In the glass furnace, the carbon monoxide and the hydrogen undergo a combustion reaction under the combustion support by the carbon-based enriched oxygen, which provides heat for the production of the molten glass in the glass furnace.

In some embodiments, after the flue gas of the glass furnace is circularly enriched for 3-5 hours, the carbon dioxide concentration reaches the equilibrium, with the carbon dioxide concentration of 95.0 v % or higher, a temperature of 1300-1400° C., and a pressure of 0.01-0.05 MPa. The circulating flue gas is divided into two ways, one way passes through the heat storage grid on the other side of the glass furnace, providing heat for the heat storage grid on the other side of the glass furnace to provide heat for the carbon-based enriched oxygen from the mixer in the next round, after heat exchange, the circulating flue gas of this way is cooled down to 550-650° C. and pressurized to 0.15-0.25 MPa, and the circulating flue gas is fed into the mixer and the second waste heat boiler; the other way does not pass through the heat storage grid of the glass furnace, with a temperature of 1300-1400° C., is pressurized to 0.5-1.0 MPa, and fed into the reforming unit.

In some embodiments, operation (7) may include following contents. The waste heat of the circulating flue gas from the glass furnace is recovered and the steam is by-produced by the second waste heat boiler. The circulating flue gas from the glass furnace has a carbon dioxide concentration of 95.0 v % or higher, a temperature of 550-650° C., and a pressure of 0.15-0.25 MPa, after waste heat recovery by the second waste heat boiler, the temperature is reduced to 250-300° C., and the pressure is 0.1-0.2 MPa, and the steam with a pressure of 1.5-4.0 MPa is by-produced for external sale. The circulating flue gas after waste heat recovery is fed into the dedusting and desulfurization unit.

In some embodiments, operation (8) may include following contents. The circulating flue gas from the second waste heat boiler is dedusted and desulfurized by the dedusting and desulfurization unit. The circulating flue gas first passes through the high-temperature dust collector of the dedusting and desulfurization unit to remove dust, and then passes through the dry desulfurization device of the dedusting and desulfurization unit to remove hydrogen sulfide. The circulating flue gas from the second waste heat boiler has a carbon dioxide concentration of 95.0 v % or higher, a temperature of 250-300° C., and a pressure of 0.1-0.2 MPa. The circulating flue gas after dust removal and desulphurization has a carbon dioxide concentration of 95.0 v % or higher, a temperature of 250-300° C., and a pressure of 0.05-0.10 MPa, a portion of which is pressurized to 2.5-3.0 MPa and fed into the methanation unit, and the rest is cooled down and sold externally as the carbon dioxide products.

In some embodiments, in the initial stage, the glass furnace adopts the natural gas as the fuel and the air as the combustion aid, and after the flue gas is produced, the carbon-based enriched oxygen mixed by the flue gas, the oxygen produced by the air separation unit, and the oxygen produced by the water electrolysis hydrogen production unit gradually replaces the air and is used as the combustion aid, and after 3-5 hours of cyclic enrichment, the carbon dioxide concentration of the circulating flue gas reaches the equilibrium, with a carbon dioxide concentration of 95.0 v % or higher, the methanation unit, the reforming unit, the first waste heat boiler, the second waste heat boiler, and the dedusting and desulfurization unit are put into operation, and the system enters into the normal operation state.

Beneficial effects of the zero-carbon glass furnace process provided by embodiments of the present disclosure include, but are not limited to as follows:
1. The embodiment of this disclosure adopts green electricity to drive the air separation unit to produce green oxygen and green nitrogen, drive the water electrolysis hydrogen production unit to produce green hydrogen and green oxygen; the flue gas of the glass furnace is circularly enriched into a high concentration of carbon dioxide (95.0 v % or higher), which then undergoes methanation reactions with green hydrogen to produce methane, methane, carbon dioxide, and water vapor undergo a reforming reaction to produce carbon monoxide and hydrogen; the carbon monoxide and hydrogen are used as the fuel of the glass furnace, the green oxygen and carbon dioxide are mixed into the carbon-based enriched oxygen as the combustion aid of the glass furnace, carbon-based enriched oxygen combustion is carried out in the glass furnace, and the excess carbon dioxide is sold externally, thereby realizing a goal of green, energy-efficient, virtually NOx-free, and zero-carbon.
2. The embodiment of the present disclosure adopts the green electricity produced by the photovoltaic power generation unit to drive the air separation unit to produce green oxygen and green nitrogen, and adopts the green electricity produced by the photovoltaic power generation unit to drive the water electrolysis hydrogen production unit to produce green hydrogen and green oxygen, which is environmentally friendly.
3. In some embodiments of the present disclosure, the flue gas of the glass furnace is circularly enriched into a gas with a carbon dioxide concentration of 95.0 v % or higher as the raw gas for methanation, the raw gas for reforming, and the raw gas for carbon-based enriched oxygen combustion of glass furnace, and the excess carbon dioxide is used as a product for sale, thereby truly realizing a zero-carbon glass furnace.
4. The embodiment of the present disclosure uses the green hydrogen produced by the water electrolysis hydrogen production unit and high-concentration carbon dioxide (95.0 v % or higher) enriched by flue gas as the raw gas for methanation to produce methane, which utilizes waste carbon dioxide and improves the utilization rate of waste resources.
5. The embodiment of the present disclosure uses methane produced by the methanation unit, high concentration of carbon dioxide (95.0 v % or higher) enriched by flue gas, and water vapor as the raw gas to produce carbon monoxide and hydrogen through the reforming reaction, which increases the calorific value of the fuel and reduces the fuel consumption.
6. The embodiment of the present disclosure adopts the high concentration of carbon dioxide (95.0 v % or higher) enriched by the flue gas to replace air for carbon-based oxygen-enriched combustion, which can make the combustion process produce virtually NOx-free, and at the same time, carbon dioxide is a greenhouse gas, which improves a thermal efficiency of the glass furnace.
7. In some embodiments of the present disclosure, the flue gas is circularly enriched into a high concentration of carbon dioxide (95.0 v % or higher), creating conditions for low-cost carbon dioxide capture and making the zero-carbon glass furnace possible.
8. The embodiment of the present disclosure combines methanation, reforming, and the glass furnace to make full use of the heat of the flue gas of the glass furnace for the heat exchange of the raw gas, improving a heat utilization rate.

The basic concepts have been described above, and it is apparent to those skilled in the art that the foregoing detailed disclosure serves only as an example and does not constitute a limitation of the present disclosure. There are various modifications, improvements, and amendments that may be made to the present disclosure by those skilled in the art. Those kinds of modifications, improvements, and amendments are suggested in the present disclosure, so that those kinds of modifications, improvements, and amendments are still within the spirit and scope of the exemplary embodiments of the present disclosure.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

What is claimed is:

1. A zero-carbon glass furnace process, wherein a system required for the process comprises a photovoltaic power generation unit, an air separation unit, a water electrolysis hydrogen production unit, a mixer, a methanation unit, a first waste heat boiler, a reforming unit, a glass furnace, a second waste heat boiler, and a dedusting and desulfurization unit; wherein
the photovoltaic power generation unit is configured to produce green electricity to supply electricity to the air separation unit and the water electrolysis hydrogen production unit;
the air separation unit is configured to produce oxygen and nitrogen;
the water electrolysis hydrogen production unit is configured to produce hydrogen and oxygen;
the mixer is configured to mix the oxygen from the air separation unit, the oxygen from the water electrolysis hydrogen production unit, and circulating flue gas from the glass furnace into carbon-based enriched oxygen;
the methanation unit is configured to methanize the hydrogen from the water electrolysis hydrogen production unit and circulating flue gas from the dedusting and desulfurization unit;
the first waste heat boiler is configured to recover waste heat of methane containing saturated water vapor from the methanation unit and by-produce steam;
the reforming unit is configured to subject the methane containing saturated water vapor from the first waste heat boiler and a portion of the circulating flue gas from the glass furnace to a methane reforming reaction;
the glass furnace is configured to produce molten glass using carbon monoxide and hydrogen from the reforming unit as fuel and the carbon-based enriched oxygen from the mixer as a combustion aid;
the second waste heat boiler is configured to recover waste heat of the circulating flue gas from the glass furnace and by-produce steam;
the dedusting and desulfurization unit is configured to dedust and desulfurize circulating flue gas from the second waste heat boiler;
the photovoltaic power generation unit is connected to the air separation unit and the water electrolysis hydrogen production unit, respectively; a nitrogen outlet of the air separation unit is connected to a tin bath of the glass furnace and to a nitrogen product storage tank, respectively, and a pressure reducing valve is provided on a connecting pipe between the nitrogen outlet of the air separation unit and the tin bath of the glass furnace; an oxygen outlet of the air separation unit and an oxygen outlet of the water electrolysis hydrogen production unit are both connected to the mixer, a pressure reducing valve is provided on a connecting pipe between the oxygen outlet of the water electrolysis hydrogen production unit and the mixer, a connecting pipe between the oxygen outlet of the air separation unit, the oxygen outlet of the water electrolysis hydrogen production unit, and the mixer is provided with a flow detector, a temperature detector, a pressure detector, an oxygen purity detector, and a flow regulating valve; a hydrogen outlet of the water electrolysis hydrogen production unit is connected to the methanation unit; a carbon-based enriched oxygen outlet of the mixer is switchably connected to a heat storage grid A/B of the glass furnace and then connected to the glass furnace; a methane outlet of the methanation unit is connected to the first waste heat boiler; a methane outlet of the first waste heat boiler is switchably connected to a heat storage grid A/B of the reforming unit and then connected to the reforming unit, a pressure reducing valve is provided on a connecting pipe between the methane outlet of the first waste heat boiler and the heat storage grid A/B of the reforming unit, and a steam outlet of the first waste heat boiler is connected to a steam utilizer; a carbon monoxide and hydrogen outlet of the reforming unit is connected to the glass furnace; one of circulating flue gas outlets of the glass furnace is switchably connected to the heat storage grid B/A of the glass furnace and then connected to the second waste heat boiler and the mixer, one of the circulating flue gas outlets of the glass furnace is switchably connected to the heat storage grid A/B of the reforming unit and then connected to the reforming unit, and one of the circulating flue gas outlets of the glass furnace is switchably connected to a heat storage grid B/A of the reforming unit and then connected to the dedusting and desulfurization unit, respectively, a pressure booster is provided on a connecting pipe between the heat storage grid B/A of the glass furnace, the second waste heat boiler, and the mixer, a connecting pipe between the heat storage grid B/A of the glass furnace and the mixer is provided with a flow detector, a temperature detector, a pressure detector, a carbon dioxide purity detector, and a flow regulating valve, a pressure booster is provided on the connecting pipe between the glass furnace, the heat storage grid A/B of the reforming unit, and the heat storage grid B/A of the reforming unit, and a pressure reducing valve is provided on a connecting pipe between the heat storage grid B/A of the reforming unit and the dedusting and desulfurization unit; a circulating flue gas outlet of the second waste heat boiler is connected to the dedusting and desulfurization unit; circulating flue gas outlets of the dedusting and desulfurization unit are connected to the methanation unit and a carbon dioxide product storage tank, respectively, and a pressure booster is provided on a connecting pipe between one of the circulating flue gas outlets of the dedusting and desulfurization unit and the methanation unit;
the process comprising operations of:
(1) the photovoltaic power generation unit producing the green electricity to supply the electricity for the air separation unit and the water electrolysis hydrogen production unit; the air separation unit producing the oxygen and the nitrogen with the green electricity supplied by the photovoltaic power generation unit as the electricity, wherein the oxygen is fed into the mixer, a portion of the nitrogen is depressurized and then fed into the tin bath of the glass furnace as protective gas, and the rest is sold outside; the water electrolysis hydrogen production unit producing the hydrogen and the oxygen with the green electricity supplied by the photovoltaic power generation unit as the electricity, wherein the hydrogen is fed into the methanation unit, and the oxygen is fed into the mixer after depressurization;
(2) the mixer mixing the oxygen from the air separation unit, the oxygen from the water electrolysis hydrogen production unit, and the circulating flue gas from the glass furnace into the carbon-based enriched oxygen, and using the carbon-based enriched oxygen as the combustion aid for the glass furnace;
(3) the methanation unit methanizing the hydrogen from the water electrolysis hydrogen production unit and the circulating flue gas from the dedusting and desulfurization unit to produce the methane containing saturated water vapor, and feeding the methane containing saturated water vapor into the first waste heat boiler;
(4) the first waste heat boiler recovering the waste heat of the methane containing saturated water vapor from the methanation unit and by-producing the steam; selling the steam externally, and feeding depressurized methane containing saturated water vapor after waste heat recovery into the reforming unit;
(5) the reforming unit subjecting the methane containing saturated water vapor from the first waste heat boiler and the portion of the circulating flue gas from the glass furnace to the methane reforming reaction; the circulating flue gas from the glass furnace entering into the reforming unit in two ways, one way entering into a heat storage grid of one side of the reforming unit, providing heat for the heat storage grid of the one side of the reforming unit to provide heat for raw gas of the reforming unit in a next round, and after heat exchange, the circulating flue gas being depressurized and fed into the dedusting and desulfurization unit; the other way entering into a heat storage grid on the other side of the reforming unit along with the methane containing saturated water vapor from the first waste heat boiler, the heat storage grid on the other side of the reforming unit being already heated by the circulating flue gas of a previous round, two sides of the heat storage grids working in rotation, and the raw gas fed into the reforming unit being heated by heat of the heat storage grid on the other side of the reforming unit, in a reaction bed of the reforming unit, under a certain pressure and a certain catalyst, methane, carbon dioxide, and water vapor undergoing the methane reforming reaction to produce the carbon monoxide and the hydrogen, and feeding the carbon monoxide and the hydrogen into the glass furnace as the fuel;
(6) the glass furnace producing the molten glass using the carbon monoxide and the hydrogen from the reforming unit as the fuel and the carbon-based enriched oxygen from the mixer as the combustion aid; the carbon-based enriched oxygen from the mixer first passing through a heat storage grid on one side of the glass furnace, the heat storage grid on the one side of the glass furnace being already heated by the previous round of the circulating flue gas, two sides of the heat storage grids working in rotation, and the carbon-based enriched oxygen from the mixer being heated to a certain temperature and then fed into the glass furnace;
after the flue gas of the glass furnace being circularly enriched for a certain period of time, a carbon dioxide concentration reaching an equilibrium; the circulating flue gas being divided into two ways, one way passing through a heat storage grid on the other side of the glass furnace, providing heat for the heat storage grid on the other side of the glass furnace to provide heat for the carbon-based enriched oxygen from the mixer in the next round, and after heat exchange, the circulating flue gas of this way being pressurized and fed into the mixer and the second waste heat boiler; the other way not passing through the heat storage grid of the glass furnace, and being pressurized and fed into the reforming unit;
(7) the second waste heat boiler recovering the waste heat of the circulating flue gas from the glass furnace and by-producing the steam; selling the steam externally, and feeding the circulating flue gas after waste heat recovery into the dedusting and desulfurization unit; and
(8) the dedusting and desulfurization unit dedusting and desulfurizing the circulating flue gas from the second waste heat boiler; pressurizing a portion of circulating flue gas after dedusting and desulfurizing and feeding pressurized circulating flue gas into the methanation unit, and selling the rest externally after cooled down as carbon dioxide products;
wherein in an initial stage, the glass furnace uses natural gas as the fuel and air as the combustion aid, and after flue gas is produced, the carbon-based enriched oxygen mixed by the flue gas, the oxygen produced by the air separation unit, and the oxygen produced by the water electrolysis hydrogen production unit gradually replaces the air and is used as the combustion aid, after a certain period of cyclic enrichment, the carbon dioxide concentration of the circulating flue gas reaches the equilibrium, and the methanation unit, the reforming unit, the first waste heat boiler, the second waste heat boiler, and the dedusting and desulfurization unit are put into operation, and the system enters into a normal operation state.

2. The process according to claim 1, wherein in operation (1), the oxygen produced by the air separation unit has a purity of 99.6 v % or higher, a normal temperature, and a pressure of 0.2-0.3 MPa, and the oxygen is fed into the mixer; the nitrogen produced by the air separation unit has a purity of 99.9 v % or higher, a normal temperature, and a pressure of 0.2-0.3 MPa, the portion of the nitrogen is depressurized to 0.05 MPa and fed into the tin bath of the glass furnace as the protective gas, and the rest is sold externally; and
the hydrogen produced by the water electrolysis hydrogen production unit has a purity of 99.8 v % or higher, a temperature of 90-100° C., and a pressure of 2.5-3.0 MPa, and the hydrogen is fed into the methanation unit; the oxygen produced by the water electrolysis hydrogen production unit has a purity of 99.0 v % or higher, a temperature of 90-100° C., and a pressure of 2.5-3.0 MPa, and the oxygen is depressurized to 0.2-0.3 MPa and fed into the mixer.

3. The process according to claim 1, wherein in operation (2), the oxygen from the air separation unit has a purity of 99.6 v % or higher, a normal temperature, and a pressure of 0.2-0.3 MPa; the oxygen from the water electrolysis hydrogen production unit has a purity of 99.0 v % or higher, a temperature of 90-100° C., and a pressure of 0.2-0.3 MPa; the circulating flue gas from the glass furnace has a carbon dioxide concentration of 95.0 v % or higher, a temperature of 550-650° C., and a pressure of 0.15-0.25 MPa; the oxygen from the air separation unit, the oxygen from the water electrolysis hydrogen production unit, and the circulating flue gas from the glass furnace are each regulated in flow rate via flow regulating valves, the carbon-based enriched oxygen has an oxygen content of 21-30 v %, a temperature of 460-480° C., and a pressure of 0.05-0.10 MPa, and the oxygen content is adjusted according to different requirements of the glass furnace.

4. The process according to claim 1, wherein in operation (3), the hydrogen from the water electrolysis hydrogen production unit has a purity of 99.8 v % or higher, a temperature of 90-100° C., and a pressure of 2.5-3.0 MPa, the circulating flue gas from the dedusting and desulfurization unit has a carbon dioxide concentration of 95.0 v % or higher, a temperature of 250-300° C., and a pressure of 2.5-3.0 MPa, the hydrogen and the circulating flue gas are fed into the methanation unit at a volume ratio of H₂ and CO₂ of 4:1, under an action of a nickel-based methanation catalyst, the hydrogen and the carbon dioxide are catalyzed to generate methane, while releasing a large amount of heat, the methane has a purity of 94.0 v % or higher on a dry basis, a temperature of 390-410° C., a pressure of 2.5-3.0 MPa, and the methane containing saturated water vapor is fed into the first waste heat boiler.

5. The process according to claim 1, wherein in operation (4), the methane containing saturated water vapor from the methanation unit has a temperature of 390-410° C., the temperature is lowered to 240-260° C. after the waste heat recovery by the first waste heat boiler and steam with a pressure of 1.5 MPa is by-produced for external sale; and the methane containing saturated water vapor after the waste heat recovery is depressurized to 0.5-1.0 MPa and then fed into the reforming unit.

6. The process according to claim 1, wherein in operation (5), the methane containing saturated water vapor from the first waste heat boiler has a methane purity of 94.0 v % or higher on a dry basis, a temperature of 240-260° C., and a pressure of 0.5-1.0 MPa, and the circulating flue gas from the glass furnace has a carbon dioxide concentration of 95.0 v % or higher, a temperature of 1300-1400° C., and a pressure of 0.5-1.0 MPa; the circulating flue gas from the glass furnace is divided into two ways to enter the reforming unit, one way enters the heat storage grid on the one side of the reforming unit, providing heat for the heat storage grid on the one side of the reforming unit to provide heat for the raw gas of the reforming unit in the next round, after the heat exchange, the temperature of the circulating flue gas is reduced to 250-300° C., and the pressure of the circulating flue gas is reduced to 0.1-0.2 MPa and then fed into the dedusting and desulfurization unit; the other way and the methane containing saturated water vapor from the first waste heat boiler are fed into the heat storage grid on the other side of the reforming unit at a volume ratio of CH₄ and CO₂ of 2:1, the heat storage grid on the other side of the reforming unit is heated by the circulating flue gas of the previous round, the two heat storage grids work in rotation, and the raw gas fed into the reforming unit is heated to 900-1000° C. by the heat storage grid on the other side of the reforming unit, under a pressure of 0.5-1.0 MPa and a nickel-based catalyst, the methane, the carbon dioxide, and the water vapor undergo the methane reforming reaction to produce the carbon monoxide and the hydrogen, with a volume ratio of CO and H₂ of 3:5, an outlet temperature of 700-800° C., and a pressure of 0.5-1.0 MPa, and the carbon monoxide and the hydrogen are fed into the glass furnace as the fuel.

7. The process according to claim 1, wherein in operation (6), the carbon monoxide and the hydrogen from the reforming unit have a volume ratio of CO and H₂ of 3:5, a temperature of 700-800° C., and a pressure of 0.5-1.0 MPa, and the carbon-based enriched oxygen from the mixer has an oxygen content of 21-30 v %, a temperature of 460-480° C., and a pressure of 0.05-0.10 MPa; the carbon-based enriched oxygen from the mixer first passes through the heat storage grid of the one side of the glass furnace, the heat storage grid is heated by the previous round of circulating flue gas, the two sides of the heat storage grids work in rotation, and the carbon-based enriched oxygen from the mixer is heated to 700-800° C. and then fed into the glass furnace; the fuel and the combustion aid have a volume ratio of 1:2; in the glass furnace, under a combustion of the carbon-based enriched oxygen, the carbon monoxide and the hydrogen undergo a combustion reaction, providing heat for the production of the molten glass in the glass furnace; and
after the flue gas of the glass furnace is circularly enriched for 3-5 hours, the carbon dioxide concentration reaches the equilibrium, with the carbon dioxide concentration of 95.0 v % or higher, a temperature of 1300-1400° C., and a pressure of 0.01-0.05 MPa; the circulating flue gas is divided into two ways, one way passes through the heat storage grid on the other side of the glass furnace, providing heat for the heat storage grid on the other side of the glass furnace to provide heat for the carbon-based enriched oxygen from the mixer in the next round, after the heat exchange, the circulating flue gas of this way is cooled down to 550-650° C. and pressurized to 0.15-0.25 MPa, and the circulating flue gas is fed into the mixer and the second waste heat boiler; the other way does not pass through the heat storage grid of the glass furnace, with a temperature of 1300-1400° C., is pressurized to 0.5-1.0 MPa, and fed into the reforming unit.

8. The process according to claim 1, wherein in operation (7), the circulating flue gas from the glass furnace has a carbon dioxide concentration of 95.0 v % or higher, a temperature of 550-650° C., and a pressure of 0.15-0.25 MPa, after the waste heat recovery by the second waste heat boiler, the temperature is reduced to 250-300° C., and the pressure is 0.1-0.2 MPa, and the steam with a pressure of 1.5-4.0 MPa is by-produced for external sale; and the circulating flue gas after the waste heat recovery is fed into the dedusting and desulfurization unit.

9. The process according to claim 1, wherein the dedusting and desulfurization unit includes a high-temperature dust collector and a dry desulfurization device; the high-temperature dust collector adopts cyclone separating and dust removing, and the dry desulfurization device adopts zinc oxide desulfurization; and
in operation (8), the circulating flue gas first passes through the high-temperature dust collector of the dedusting and desulfurization unit to remove dust, and then passes through the dry desulfurization device of the dedusting and desulfurization unit to remove hydrogen sulfide; the circulating flue gas from the second waste heat boiler has a carbon dioxide concentration of 95.0 v % or higher, a temperature of 250-300° C., and a pressure of 0.1-0.2 MPa, and the circulating flue gas after dust removal and desulphurization has a carbon dioxide concentration of 95.0 v % or higher, a temperature of 250-300° C., and a pressure of 0.05-0.10 MPa, a portion of which is pressurized to 2.5-3.0 MPa and fed into the methanation unit, and the rest is cooled down and sold externally as the carbon dioxide products.

10. The process according to claim 1, wherein in the initial stage, the glass furnace adopts the natural gas as the fuel and the air as the combustion aid, and after the flue gas is produced, the carbon-based enriched oxygen mixed by the flue gas, the oxygen produced by the air separation unit, and the oxygen produced by the water electrolysis hydrogen production unit gradually replaces the air and is used as the combustion aid, and after 3-5 hours of cyclic enrichment, the carbon dioxide concentration of the circulating flue gas reaches the equilibrium, with a carbon dioxide concentration of 95.0 v % or higher, the methanation unit, the reforming unit, the first waste heat boiler, the second waste heat boiler, and the dedusting and desulfurization unit are put into operation, and the system enters into the normal operation state.
